(19) United States
(12) Patent Application Publication
(10) Pub. No.: US 2016/0095808 A1
(43) Pub. Date: Apr. 7, 2016

(54) HAIR CARE COMPOSITION COMPRISING DISCRETE PARTICLE OF OILY COMPONENT

(71) Applicant: The Procter & Gamble Company, Cincinnati, OH (US)

(72) Inventors: Toshiyuki OKADA, Singapore (SG); Raymond Xu Zhan TAY, Singapore (SG); Qing STELLA, Cincinnati, OH (US)

(21) Appl. No.: 14/873,654

(22) Filed: Oct. 2, 2015

Related U.S. Application Data
(60) Provisional application No. 62/059,505, filed on Oct. 3, 2014.

(51) Int. Cl. A61K 8/73; A61K 8/86; A61K 8/97; A61K 8/92; A61Q 5/12; A61Q 5/06; A61Q 5/02; A61K 8/02; A61K 8/34

(52) U.S. Cl. CPC A61K 8/73; A61K 8/0216; A61K 8/86; A61K 8/97; A61K 8/345; A61Q 5/12; A61Q 5/06; A61Q 5/02; A61K 8/92; A61K 2800/412; A61K 2800/49; A61K 2800/5422; A61K 2800/524; A61K 2800/596

(57) ABSTRACT

Disclosed is a hair care composition comprising: a discrete particle comprising an oily component, wherein the oily component comprises one or more materials selected from the group consisting of: (A) metathesized unsaturated polyol esters; (B) sucrose polyesters; (C) fatty esters with a molecular weight greater than or equal to 1500; and mixtures thereof, wherein the oily component has a Zero Shear Viscosity at 25° C. of from about 10² Pa·s to about 10⁹ Pa·s, and has a melting point of from about 35° C. to about 60° C.; and wherein the discrete particle has an average particle size in the hair care composition of from about 0.5 microns to about 20 microns. The hair care composition provides improved conditioning benefits, clean feel, and/or hair styling benefit.

### FIELD OF THE INVENTION

The present invention relates to Disclosed is a hair care composition comprising: a discrete particle comprising an oily component, wherein the oily component comprises one or more materials selected from the group consisting of: (A) metathesized unsaturated polyol esters; (B) sucrose polyesters; (C) fatty esters with a molecular weight greater than or equal to 1500; and mixtures thereof, wherein the oily component has a Zero Shear Viscosity at 25° C. of from about 10² Pa·s to about 10⁹ Pa·s, and has a melting point of from about 35° C. to about 60° C.; and wherein the discrete particle has an average particle size in the hair care composition of from about 0.5 microns to about 20 microns. The hair care composition provides improved conditioning benefits, clean feel, and/or hair styling benefit.

### BACKGROUND OF THE INVENTION

Human hair becomes soiled due to its contact with the surrounding environment and from the sebum secreted by the scalp. The soiling of hair causes it to have a dirty feel and an unattractive appearance.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils.

A variety of approaches have been developed to alleviate these after-shampoo problems. One approach is the application of a conditioner after shampooing.

In order to provide hair conditioning benefits after shampooing, a wide variety of conditioning actives have been proposed. These conditioning agents are known to enhance hair shine and provide moistness, softness, and static control to the hair. However, such components can also provide stickiness, greasy, or waxy feeling, particularly when the hair is dried.

Silicone conditioning agents are also known to provide conditioning benefits such as smoothness and combing ease due to the low surface tension of silicone compounds. However, silicone conditioning agents can cause dry feel or frizzy condition to the hair, again, particularly when the hair is dried. Additionally, the rising costs and the petroleum based nature of silicone have minimized silicone's desirability as a conditioning active.

Based on the foregoing, there has been a need for a conditioning active which can provide conditioning benefits to hair and can replace, or be used in combination with silicone, or other conditioning actives, to maximize the conditioning activity of hair care compositions.

For example, US 2009/0220443 A1 discloses a petrolatum-like composition comprising a metathesized unsaturated polyol ester, which emulsions may be used, for example, as replacements for petroleum-derived wax emulsions in a variety of applications including cosmetic and personal care formulations including hair care products. The US publication discloses in [0010] that, in an embodiment, the petrolatum-like composition comprises about 5% wt. to about 25% wt. hydrogenated metathesized soybean oil, and about 75% wt. to about 95% wt. soybean oil. As for the particle size, the US publication also describes in [0095] that, in some embodiments, the dispersed phase of the emulsion has a particle size of about 1 micron or less, and also in [0104] that emulsion is stable at about 0.1 to 1.5 micron, and the particle size promotes thorough, homogeneous incorporation with other ingredients that may be present in a formulation comprising the emulsion.

However, when incorporating such a petrolatum-like composition in a hair care composition, there still is a need to improve conditioning benefits, clean feel, and/or hair styling benefit.

### SUMMARY OF THE INVENTION

The present invention is directed to a hair care composition comprising:

a discrete particle comprising an oily component,
wherein the oily component comprises one or more materials selected from the group consisting of:
(A) metathesized unsaturated polyol esters;
(B) sucrose polyesters;
(C) fatty esters with a molecular weight greater than or equal to 1500; and mixtures thereof,
   wherein the oily component has a Zero Shear Viscosity at 25° C. of from about 10² Pa·s to about 10⁹ Pa·s, and has a melting point of from about 35° C. to about 60° C.; and
   wherein the discrete particle has an average particle size in the hair care composition of from about 0.5 microns to about 20 microns.

The hair care composition of the present invention provides improved conditioning benefits, clean feel, and/or hair styling benefit. Clean feel herein includes, for example, less oily/greasy feel and/or less coated feel.

The present invention is also directed to a method of providing hair styling benefit to hair, comprising a step of applying to the hair an effective amount of the above hair care composition.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The terms “include,” “includes,” and “including,” as used herein, are meant to be non-limiting and are understood to mean “comprise,” “comprises,” and “comprising,” respectively.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated. The term “weight percent” may be denoted as “wt. %” herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Discrete Particle

The hair care composition of the present invention comprises a discrete particle of an oily component. Details of the oily components are explained below under the title “OILY COMPONENT”. The oily component can be added to the hair care composition with or without pre-emulsification to form the discrete particle in the hair care composition.

The discrete particle has an average particle size in the hair care composition of from about 0.5 microns to about 20 microns. Such average particle size is preferably from about 0.8 microns, more preferably from about 1.0 microns, and preferably to about 15 microns, more preferably to about 10 microns, still more preferably to about 6.0 microns, in view of providing the benefits of the present invention.

The inventors of the present invention have found that, even if pre-emulsion is formed for the oily components to have a certain particle size, such particle size changes when incorporating the pre-emulsion in the hair care composition. Then, the inventors of the present invention have surprisingly found that, the specific particles size in the hair care composition is more important to provide the benefits of the present invention, compared to the particle size of pre-emulsion.

Preferably, the discrete particle has a certain particle size distribution in the hair care composition while having the above average particle sizes. Such particle size distribution in the hair care composition is as follows: D99.5 is preferably 15 micron or less, more preferably 12 micron or less, and preferably 0.5 micron or more, more preferably 0.8 micron or more. The inventors of the present invention have also surprisingly found that, a certain particle size distribution contributes to provide further improved benefits of the present invention, especially clean feel such as less coated feel.

The particle size of the discrete particle in hair care composition described herein is quantified by the following measurement methodology. The method involves an image capturing device and 3 softwares. Keyence reflected light microscope or any reflected light microscope could be used as the image capturing device. Microsoft Office Picture Manager, ImageJ and JMP Pro 11 are softwares used in this method. The parameters and methodology used are described below. The sample is prepared on a glass slide and a microscope image is taken using Keyence reflected light microscope. Image taken is pre-processed by Microsoft Office Picture Manager to convert it to monochrome. Preferred setting is as follows: Saturation −100, Brightness 10, Contrast 70, Midtones 10. The image is then processed and analyzed by ImageJ. Image is processed by ImageJ using Fourier transform to minimize the noise and to set threshold on objects to be measured. Preferred setting is as follows: FFT bandpass filter <3 pixels, Threshold 120. The image is then edited by manually closing the breakages in the outlines of the particles. The image is then analyzed by ImageJ to get the particle size of the discrete particle and their counts. The output is then processed by JMP Pro 11 to get the histogram of the distribution, average particle size and D99.5 values.

### Pre-Emulsion

Preferably in the present invention, the oily component is pre-emulsified before adding to the hair care composition, and added to the hair care composition in a form of pre-emulsion. Without being limited by theory, it is believed that the use of the pre-emulsion provides better control of particle size of the discrete particles in hair care composition, and also provides improved homogeneity of the hair care composition. Thus, without being limited by theory, it is believed that the use of the pre-emulsion contributes to provide further improved benefits of the present invention.

Pre-emulsion can have a particle size of preferably from about 0.02 microns to about 100 microns, more preferably from about 0.1 microns to about 50 microns, still more preferably from about 0.5 microns to about 10 microns, even more preferably from about 0.5 microns to about 6.0 microns. The particle size of the pre-emulsion can be measured by any conventional particle size measuring methodology, for example, by using Horiba LA-910 Laser scattering particle size distribution analyzer.

Hair care composition may comprise from about 0.25% to about 80% of a pre-emulsion. When hair care composition comprising cationic surfactants, high melting point fatty compounds and aqueous carrier, the pre-emulsion can be incorporate at a level by weight of the hair care composition, preferably from about 0.25% to about 50%, more preferably from about 0.5% to about 20%, still more preferably from about 1% to about 15%.

Such pre-emulsion further contains an emulsifier, and may also contain a carrier. Such emulsifiers and carriers are explained below.

### Emulsifiers

The concentration of the emulsifier in the pre-emulsion should be sufficient to provide the desired emulsification of the oily component, and generally ranges for example, from about 0.1 wt. % to about 20 wt %, preferably from about 0.2 wt. % to about 10 wt. %, more preferably from about 0.3 wt. % to about 7 wt. %, still more preferably 0.5 wt % to about 3% by weight of the pre-emulsion, in view of better compatibility with cationic surfactant system and/or gel matrix which are preferably used in the composition, while providing desired emulsification.

Emulsifiers useful herein include non-ionic, cationic, anionic and amphoteric emulsifiers. In view of better compatibility with cationic surfactant system and/or gel matrix which are preferably used in the composition, nonionic and/or cationic emulsifiers are preferably used in the pre-emulsion. More preferably, nonionic emulsifiers are used in the pre-emulsion, and the pre-emulsion is free of other emulsifiers, i.e., does not contain other emulsifiers. By the emulsion being free of other emulsions, it is believed that: the emulsion may have further better compatibility with cationic surfactant system and/or gel matrix which are preferably used in the composition; and/or it may become easier to control the particle size of discrete particles in the hair care composition.

Non-ionic emulsifiers suitable for use in the emulsion may include a wide variety of emulsifiers useful herein and include, but not limited to, those selected from the group consisting of sorbitan esters, glyceryl esters, polyglyceryl esters, methyl glucose esters, sucrose esters, ethoxylated fatty alcohols, hydrogenated castor oil ethoxylates, sorbitan ester ethoxylates (polysorbates), polymeric emulsifiers, and silicone emulsifiers.

Among a variety of nonionic surfactants, sorbitan esters and sorbitan ester ethoxylates (polysorbates) are preferably used, and sorbitan ester ethoxylates (polysorbates) are more preferably used. Further preferred are liquid at 25° C. and having an HLB of from about 7 to about 16.

Such sorbitan esters are, for example, sorbitan esters of C16-C22 saturated, unsaturated and branched-chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN(Registered trademark) 80), sorbitan sesquioleate (e.g., Arlacel(Registered trademark) 83), sorbitan monoisostearate (e.g., CRILL(Registered trademark) 6 made by Croda), sorbitan stearates (e.g., SPAN(Registered trademark) 60), sorbitan triooleate (e.g., SPAN(Registered trademark) 85), sorbitan tristearate (e.g., SPAN(Registered trademark) 65), sorbitan dipalmitates (e.g., SPAN(Registered trademark) 40), and sorbitan isostearate. Sorbitan monoisostearate and sorbitan sesquioleate are particularly preferred among them.

Such sorbitan ester ethoxylates (polysorbates) are, for example, Polysorbate 20 (Polyoxyethylene (20) sorbitan monolaurate), Polysorbate 40 (Polyoxyethylene (20) sorbitan monopalmitate), Polysorbate 60 (Polyoxyethylene (20) sorbitan monostearate), and Polysorbate 80 (Polyoxyethylene (20) sorbitan monooleate). Among them, highly preferred is Polysorbate 20 (Polyoxyethylene (20) sorbitan monolaurate).

Cationic emulsifiers suitable for use in the emulsion of the present invention may include a wide variety of emulsifiers. Such cationic emulsifiers include, for example, those described below under the title “CATIONIC SURFACTANT SYSTEM”.

### Carrier

The pre-emulsion may contain a carrier such as an aqueous carrier, more specifically water. Generally, the pre-emulsion comprise from about 20% to about 99%, preferably from about 30% to about 95%, and more preferably from about 80% to about 95% carrier by weight of the pre-emulsion.

The carrier can also contain a polyol such as glycerin. Polyol useful herein are those having a molecular weight of from about 40 to about 500, preferably from about 50 to about 350, more preferably from about 50 to about 200, still more preferably from about 50 to about 150. Polyols useful herein are preferably water soluble, and include, for example: pentaerythritol; propylene glycol; butylene glycol; glycerin; pentylene glycol; hexylene glycol; Diols such as 1,2-diol, 1,3-diol, and other diols, the diols having a hydrocarbon chain having 1-20 carbons, preferably 1-6 carbons; polyethylene glycol; polypropylene glycol; polybutylene glycol; polypentylene glycol; and polyhexylene glycol. Among them, preferred are Glycerin, Butylene Glycol, Propylene glycol, more preferred are glycerin. Polyols can be included in the pre-emulsion up to about 50%.

### Oily Component

The hair care composition comprises an oily component comprising one or more materials selected from the group consisting of (A) metathesized unsaturated polyol esters, (B) sucrose polyesters, (C) fatty esters with a molecular weight greater than or equal to 1500, and mixtures thereof. The details of such materials (A), (B), and (C) are explained later.

The hair care composition comprises such oily component at a level of from about 0.005% to about 20%, preferably from about 0.01% to about 15%, more preferably from about 0.03% to about 10%, still more preferably from about 0.03% to about 5%, by weight of the hair care composition.

The hair care composition comprises such one or more material at a level of from about 0.005% to about 5%, preferably from about 0.01% to about 3%, more preferably from about 0.01% to about 2%, still more preferably from about 0.01% to about 1% by weight of the hair care composition.

The oily component useful herein has a Zero Shear Viscosity at 25° C. of from about 10² Pa·s to about 10⁹ Pa·s, preferably from about 10³ Pa·s to about 10⁸ Pa·s, more preferably from about 10⁴ Pa·s to about 10⁷ Pa·s.

The oily component useful herein has a melting point of from about 35° C. to about 60° C., preferably from about 40° C. to about 52° C., more preferably from about 40° C. to about 50° C.

Preferably, especially when the above material (A), (B) and/or (C) has higher melting point and/or higher viscosity, the oily component further comprises a liquid oily material which is a liquid at 25° C. so that the mixture of the above material and the liquid oily material has the above Zero Shear Viscosity and melting point. Such liquid oily material is preferably miscible and non-reactive to the material (A), (B) and/or (C). Such liquid oily material is preferably vegetable oils such as sesame oil, canola oil, soy bean oil. Synthetic oils such as silicone oils and paraffin oils may be also used as such liquid oily materials.

When the oily component comprises the material (A) a metathesized unsaturated polyol ester, it is preferred that the oily component further comprises a liquid oily material being a non-metathesized unsaturated polyol ester. More specifically, when the oily component comprises the material (A) a metathesized unsaturated polyol ester being a soy oligomer, it is preferred that the oily component further comprises a liquid oily material which is non-metathesized unsaturated polyol ester being a soybean oil. In this case, the weight ratio of the liquid oily material to the material (A) is preferably from about 98:2 to about 70:30, more preferably from about 95:5 to about 75:25, still more preferably from about 95:5 to about 80:20.

### Yield Stress, Young's Modulus, and Zero Shear Viscosity Method

The Yield Stress and Zero Shear Viscosity can be measured by the following method.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First a sample is obtained and placed in position on the rheometer base plate, the measurement geometry (upper plate) moving into position 1 mm above the base plate. Excess sample at the geometry edge is removed by scraping after locking the geometry. If the sample comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a sample has any particle larger than 5 mm in any dimension, the particles are removed prior to the measurement.

The determination is performed via the programmed application of a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure.

The Young's Modulus (Pa) is obtained by graphing the Stress (Pa) vs. Strain (unitless). Young's modulus is derived from the slope of the regression line of the initial linear region between Stress vs. Strain graph. Structured surfactant compositions in the present invention typically exhibit linear region in the strain range of 0 to about 0.05.

The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### (A) Metathesized Unsaturated Polyol Ester

Exemplary metathesized unsaturated polyol esters and their starting materials are set forth in U.S. Patent Application U.S. 2009/0220443 A1, which is incorporated herein by reference.

A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds (i.e., olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I:

R¹—CH═CH—R²+R¹—CH═CH—R² [symbol] R¹—CH═CH—R¹+R²—CH═CH—R² (I)

where R¹ and R² are organic groups.

Cross-metathesis may be represented schematically as shown in Equation II:

R¹—CH═CH—R²+R³—CH═CH—R⁴ [symbol] R¹—CH═CH—R³+R—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴ (II)

where R¹, R², R³, and R⁴ are organic groups.

When the unsaturated poyol ester comprises molecules that have more than one carbon-carbon double bond (i.e., a polyunsaturated polyol ester), self-metathesis results in oligomerization of the unsaturated polyol ester. The self-metathesis reaction results in the formation of metathesis dimers, metathesis trimers, and metathesis tetramers. Higher order metathesis oligomers, such as metathesis pentamers and metathesis hexamers, may also be formed by continued self-metathesis and will depend on the number and type of chains connecting the unsaturated polyol ester material as well as the number of esters and orientation of the ester relative to the unsaturation

As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. In many embodiments, the unsaturated polyol ester can be represented by the general structure I:

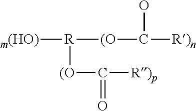

where n≧1; m≧0; p≧0; (n+m+p)≧2; R is an organic group; R′ is an organic group having at least one carbon-carbon double bond; and R″ is a saturated organic group. Exemplary embodiments of the unsaturated polyol ester are described in detail in U.S. 2009/0220443 A1.

In many embodiments of the invention, the unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of these, and the like. Recycled used vegetable oils may also be used. Representative examples of vegetable oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high erucic rape oils, Jatropha oil, combinations of these, and the like. Representative examples of animal fats include lard, tallow, chicken fat, yellow grease, fish oil, combinations of these, and the like. A representative example of synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Sugar esters such as SEFOSE® include one or more types of sucrose polyesters, with up to eight ester groups that could undergo a metathesis exchange reaction. Such sucrose polyesters include, for example, those described below under the title “(B) SUCROSE POLYESTERS”.

Other examples of suitable natural polyol esters may include but not be limited to sorbitol esters, maltitol esters, sorbitan esters, maltodextrin derived esters, xylitol esters, and other sugar derived esters.

In other embodiments, chain lengths of esters are not restricted to C8-C22 or even chain lengths only and can include natural esters that come from co-metathesis of fats and oils with short chain olefins both natural and synthetic providing a polyol ester feedstock which can have even and odd chains as well as shorter and longer chains for the self-metathesis reaction. Suitable short chain olefins include ethylene and butene.

Oligomers Derived from Metathesis of Unsaturated Polyol Esters

Oligomers derived from the metathesis of unsaturated polyol esters are preferably used. Oligomers useful herein include, for example, dimer, trimer, tetramer, pentamer, and/or hexamer, preferably, dimer, trimer, and/or tetramer, more preferably, a mixture of dimer, trimer, and/or tetramer.

The oligomers may be further modified via hydrogenation. For example, in certain embodiments, the oligomer can be about 60% hydrogenated or more; in certain embodiments, about 70% hydrogenated or more; in certain embodiments, about 80% hydrogenated or more; in certain embodiments, about 85% hydrogenated or more; in certain embodiments, about 90% hydrogenated or more; and in certain embodiments, generally 100% hydrogenated.

In some embodiments, the triglyceride oligomer is derived from the self-metathesis of soybean oil. The soy oligomer can include hydrogenated soy polyglycerides. The soy oligomer may also include C₁₅-C₂₃ alkanes, as a byproduct. An example of metathesis derived soy oligomers which is hydrogenated is available from Elevance as CS-110.

In other embodiments, the metathesized unsaturated polyol esters can be used as a blend with one or more non-metathesized unsaturated polyol esters. The non-metathesized unsaturated polyol esters can be fully or partially hydrogenated. Such an example is available from Elevance as CG-100, a blend of CS-110 oligomer and hydrogenated soybean oil (HSBO). In some embodiments of the invention, the non-metathesized unsaturated polyol ester is an unsaturated ester of glycerol. Sources of unsaturated polyol esters of glycerol include synthesized oils, natural oils (e.g., vegetable oils, algae oils, bacterial-derived oils, and animal fats), combinations of theses, and the like. Recycled used vegetable oils may also be used. Representative examples of vegetable oils include those listed above.

Other modifications of the polyol ester oligomers can be partial amidation of some fraction of the esters with ammonia or higher organic amines such as dodecyl amine or other fatty amines. This modification will alter the overall oligomer composition but can be useful in some applications providing increased lubricity of the product. Another modification can be via partial amidation of a poly amine providing potential for some pseudo cationic nature to the polyol ester oligomers. Other exemplary embodiments of amido functionalized oligomers are described in detail in WO2012006324A1, which is incorporated herein by reference.

The poloyl ester oligomers may be modified further by partial hydroformylation of the unsaturated functionality to provide one or more OH groups and an increase in the oligomer hydrophilicity.

In other embodiments, the unsaturated polyol esters and blends can be modified prior to oligomerization to incorporate near terminal branching. Exemplary polyol esters modified prior to oligomerization to incorporate terminal branching are set forth in WO2012/009525 A2, which is incorporated herein by reference.

### (B) Sucrose Polyester

Typical examples of sucrose polyesters such as SEFOSE®. The sucrose molecule can be esterified in one or more of its eight hydroxyl groups with saturated or unsaturated carboxylic acids, providing a very diverse set of possible molecular structures of polyesters. The possibility of metathesis of these species was described above under the title “(A) METATHESIZED UNSATURATED POLYOL ESTER”. However, as explained herein, non-metathesized unsaturated sucrose polyesters, saturated sucrose polyesters, and/or their mixtures can also be used.

Sucrose polyesters are derived from a natural resource and therefore, the use of sucrose polyesters can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. In one embodiment the sucrose polyester may have an IBAR of from about 5 to about 8. In another embodiment the sucrose polyester has an IBAR of about 5-7, and in another embodiment the sucrose polyester has an IBAR of about 6. In yet another embodiment the sucrose polyester has an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140. In another embodiment the sucrose polyester may have an IV of about 10 to about 120. In yet another embodiment the sucrose polyester may have an IV of about 20 to 100. Further, such sucrose polyesters have a chain length of about C₁₂ to C₂₀ but are not limited to these chain lengths.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

(C) Fatty Esters with Higher Molecular Weight

Fatty esters useful herein are those with a molecular weight greater than or equal to 1500. Conventional fatty esters having such molecular weight can be used.

### Cationic Surfactant System

The composition of the present invention preferably comprises a cationic surfactant system. The cationic surfactant system can be one cationic surfactant or a mixture of two or more cationic surfactants. Preferably, the cationic surfactant system is selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt. More preferably, the cationic surfactant system is a mixture of mono-long alkyl quaternized ammonium salt and di-long alkyl quaternized ammonium salt.

The cationic surfactant system can be included in the composition at a level by weight of from about 0.1% to about 10%, preferably from about 0.5% to about 8%, more preferably from about 0.8% to about 5%, still more preferably from about 1.0% to about 4%.

### Mono-Long Alkyl Quaternized Ammonium Salt

The mono-long alkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

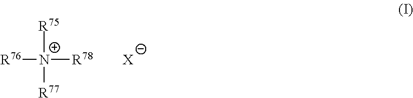

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

### Di-Long Alkyl Quaternized Ammonium Salt

Di-long alkyl quaternized ammonium salt is, when used in the composition, preferably combined with a mono-long alkyl quaternized ammonium salt or mono-long alkyl amidoamine salt. It is believed that such combination can provide easy-to rinse feel, compared to single use of a mono-long alkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the di-long alkyl quaternized ammonium salt in the cationic surfactant system is in the range of preferably from about 10% to about 50%, more preferably from about 30% to about 45%.

The di-long alkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

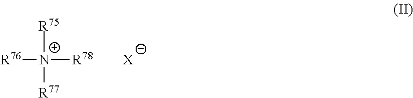

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such di-long alkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound can be included in the composition at a level of from about 0.1% to about 20%, preferably from about 1% to about 15%, more preferably from about 1.5% to about 8% by weight of the composition, in view of providing improved conditioning benefits such as slippery feel during the application to wet hair, softness and moisturized feel on dry hair.

### Aqueous Carrier

The composition of the present invention preferably comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

### Gel Matrix

Preferably, in the present invention, a gel matrix is formed by the cationic surfactant, the high melting point fatty compound, and an aqueous carrier. When such gel matrix is contained, the discrete particles of the oily components are dispersed in such gel matrix. The gel matrix is suitable for providing various conditioning benefits, such as slippery feel during the application to wet hair and softness and moisturized feel on dry hair.

Preferably, when the gel matrix is formed, the cationic surfactant and the high melting point fatty compound are contained at a level such that the weight ratio of the cationic surfactant to the high melting point fatty compound is in the range of, preferably from about 1:1 to about 1:10, more preferably from about 1:1.5 to about 1:7, still more preferably from about 1:2 to about 1:6, in view of providing improved wet conditioning benefits.

Preferably, when the gel matrix is formed, the composition of the present invention is substantially free of anionic surfactants, in view of stability of the gel matrix. In the present invention, “the composition being substantially free of anionic surfactants” means that: the composition is free of anionic surfactants; or, if the composition contains anionic surfactants, the level of such anionic surfactants is very low. In the present invention, a total level of such anionic surfactants, if included, preferably 1% or less, more preferably 0.5% or less, still more preferably 0.1% or less by weight of the composition. Most preferably, the total level of such anionic surfactants is 0% by weight of the composition.

### Silicone Compound

The compositions of the present invention may contain a silicone compound. The silicone compounds are included at levels by weight of the composition of from about 0.05% to about 15%, preferably from about 0.1% to about 10%, more preferably from about 0.1% to about 8%.

Preferably, the silicone compounds have an average particle size of from about 1 microns to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

In some embodiments, amino substituted silicones are preferably used. Preferred aminosilicones include, for example, those which conform to the general formula (I):

(R₁)ₐG₃₋ₐ-Si—(—OSiG₂)ₙ-(—OSiG_{b}(R₁)_{2-b})ₘ—O-SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions.

These include, for example, conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithione.

### Product Forms

The compositions of the present invention can be in the form of rinse-off products or leave-on products, and can be formulated in a wide variety of product forms, including but not limited to creams, gels, emulsions, mousses and sprays. The hair care compositions of the present invention can be used as a wide variety of hair care products, including but not limited to hair conditioning products, hair treatment products, and hair styling products.

The composition of the present invention is especially suitable for rinse-off hair conditioner. Such compositions are preferably used by following steps:

(i) after shampooing hair, applying to the hair an effective amount of the conditioning compositions for conditioning the hair; and
(ii) then rinsing the hair.

### Styling Benefit

The present invention also directed to a method of providing hair styling benefit to hair, comprising a step of applying to hair an effective amount of the above hair care composition.

Such styling benefits are, for example, easy to style and/or keep style. Easy to style includes, for example, easy to comb/detangling when dry, easy to manage hair tips, easy to style, easy to style using heated tools, and/or easy to create curl. Keep style includes, for example, keep style longer, keeps hair feeling clean longer and/or not weighing hair down. Some of the above benefits can be translated to another benefit, for example, easy to comb/detangling when dry can be translated to conditioning benefit as well as easy to style benefit, and keeps hair feeling clean longer can be translated to clean benefit as well as keep style benefit.

Effective amount herein is, for example, from about 0.1 ml to about 2 ml per 10 g of hair, preferably from about 0.2 ml to about 1.5 ml per 10 g of hair.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1
[Pre-Emulsion examples]

| Ingredient | Ex. A | Ex. B |
|---|---|---|
| Distilled Water | q.s. | q.s. |
| Polysorbate-20 *1 | 0.66 | 1.91 |
| Monoglyceride *2 | 0.34 | 0.97 |
| Soy Oligomer *3 | — | 10.0 |
| Blend of 10% Soy Oligomer | 20.0 | — |
| and 90% Soybean oil *4 | | |
| Glycerine | 45.0 | 7.5 |
| Preservatives, pH, viscosity | Up to 3% | Up to 3% |
| adjustment | | |
| Average particle size | About 1.3 microns | About 0.7 microns |

TABLE 2
Hair care composition examples

| Components | Ex. 1 | Ex. 2 | Ex. 3 | Ex. 4 | CEx. i | CEx. ii |
|---|---|---|---|---|---|---|
| Pre-emulsion Ex. A | 2.5 (0.5) | 5.0 (1.0) | 2.5 (0.5) | 2.5 (0.5) | — | — |
| (% of a blend of 10% Soy | | | | | | |
| oligomer and 90% Soybean oil, | | | | | | |
| in hair care composition) | | | | | | |
| Pre-emulsion Ex. B | — | — | — | — | 5.0 (0.5) | — |
| (% of Soy oligomer in hair care | | | | | | |
| composition) | | | | | | |
| Blend of 10% Soy Oligomer and | — | — | — | — | — | 0.5 |
| 90% Soybean oil *4 | | | | | | |
| Stearamidopropyldimethylamine | 1.06 | 1.06 | 1.06 | 1.06 | 1.06 | 1.06 |
| l-glutamic acid | 0.34 | 0.34 | 0.34 | 0.34 | 0.34 | 0.34 |
| Behenyl trimethyl ammonium | — | — | — | — | — | — |
| methosulfate | | | | | | |
| Dicetyl dimethyl ammonium | 0.5 | 0.5 | 0.5 | 0.5 | 0.5 | 0.5 |
| chloride | | | | | | |
| Cetyl alcohol | 1.5 | 1.5 | 1.5 | 1.5 | 1.5 | 1.5 |
| Stearyl alcohol | 2.8 | 2.8 | 2.8 | 2.8 | 2.8 | 2.8 |
| Aminosilicone *5 | — | — | — | 0.5 | — | — |
| Preservatives | 0.43 | 0.43 | 0.43 | 0.43 | 0.43 | 0.43 |
| Perfume | 0.5 | 0.5 | 0.5 | 0.5 | 0.5 | 0.5 |
| Deionized Water | q.s. to 100% | | | | | |
| Average particle size of the | About 2.5 | About 2.5 | About 3.0 | About 2.5 | About 50 | About 0.4 |
| discrete particle of the oily | micron | micron | micron | micron | micron | micron |
| component in the hair care | | | | | | |
| composition | | | | | | |
| Particle size distribution of the | <11.0 | — | — | — | — | — |
| discrete particle of the oily | microns | | | | | |
| component in the hair care | | | | | | |
| composition (D99.5) | | | | | | |
| Zero Shear Viscosity of the oily | 2.3 × 10⁵ | 2.3 × 10⁵ | 2.3 × 10⁵ | 2.3 × 10⁵ | Over 10⁹ | 2.3 × 10⁵ |
| component at 25° C. | Pa · s | Pa · s | Pa · s | Pa · s | Pa · s (Too | Pa · s |
| | | | | | thick to measure) | |
| Melting point of the oily | 47-49° C. | 47-49° C. | 47-49° C. | 47-49° C. | 55° C. | 47-49° C. |
| component | | | | | | |

### DEFINITIONS OF COMPONENTS

*1 Tween 20, from Lonza
*2 Glyceryl monooleate, from BASF
*3 CS-110, from Elevance
*4 CG-100, from Elevance
*5 Aminosilicone: Terminal aminosilicone which is available from GE having a viscosity of about 10,000 mPA·s, and having following formula:

(R₁)ₐG₃₋ₐ-Si—(—OSiG₂)ₙ-O—SiG₃₋ₐ(R₁)ₐ
wherein G is methyl; a is an integer of 1; n is a number from 400 to about 600; R₁ is a monovalent radical conforming to the general formula C_{q}H_{2q}L, wherein q is an integer of 3 and L is —NH₂.

### Method of Preparation

The hair care compositions of “Ex. 1” through “Ex. 4”, “CEx.i” and “CEx.ii” as shown above can be prepared by any conventional method well known in the art.

### Properties and Benefits

For some of the compositions, properties and benefits are evaluated by the following methods. Such properties and benefits of the compositions and results of the evaluation are shown above.

Examples 1 through 4 are hair care compositions of the present invention, which are particularly useful as rinse-off hair conditioning compositions. After shampooing hair, an effective amount of the hair care compositions are applied to the hair, and then rinsed off.

The embodiments disclosed and represented by the previous “Ex. 1” through “Ex. 4” have many advantages. For example, they provide improved conditioning benefits, clean feel, and/or hair styling benefit.

Such advantages can be understood by the comparison between the examples of the present invention and comparative examples “CEx.i” and “CEx.ii”, for example, by the comparison between Ex. 1 and these comparative examples in below panelist test.

### Panelist Test

Conditioning benefits, clean feel, and/or hair styling benefit are evaluated by a panelist test. For each hair care composition, about 200 panelists joined in the test. The panelists use one hair care composition as a rinse-off conditioner after shampooing, then, choose one description which matches the hair care composition among 5 different levels of satisfactions (such as “very good”, “fairly good”, “average”, “somewhat poor”, and “very poor”) for each hair condition in below table. The data from the panelist group testing the same composition are corrected, averaged and scored, then compared to an average score from another panelist group testing another composition.

Such score comparisons per each hair condition are shown in below table.

| Hair conditions | Average score from panelist groups |
|---|---|
| Easy to comb/detangling when | Panelist group for Ex. 1 put a better score than other panelist |
| dry | groups for CEx. i and CEx. ii |
| Easy to manage hair tips | Panelist group for Ex. 1 put a better score than other panelist |
| | groups for CEx. i and CEx. ii |
| Keep style longer | Panelist group for Ex. 1 put a better score than the panelists |
| | group for CEx. i, and a slightly better score than the panelists |
| | group for CEx. ii |
| Leaves hair with body and | Panelist group for Ex. 1 put a better score than other panelist |
| fullness | groups for CEx. i and CEx. ii |
| Helps my heated styling tool to | Panelist group for Ex. 1 put a much better score than other |
| work better | panelist groups for CEx. i and CEx. ii |
| Keeps hair feeling clean longer | Panelist group for Ex. 1 put a better score than the panelists |
| | group for CEx. i, and a slightly better score than the panelists |
| | group for CEx. ii |
| Not weighing hair down | Panelist group for Ex. 1 put a slightly better score than the |
| | panelists group for CEx. i, and a better score than the panelists |
| | group for CEx. ii |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a discrete particle comprising an oily component,
wherein the oily component comprises one or more materials selected from the group consisting of:
(A) metathesized unsaturated polyol esters;
(B) sucrose polyesters;
(C) fatty esters with a molecular weight greater than or equal to 1500; and mixtures thereof,
wherein the oily component has a Zero Shear Viscosity at 25° C. of from about 10² Pa·s to about 10⁹ Pa·s, and has a melting point of from about 35° C. to about 60° C.; and
wherein the discrete particle has an average particle size in the hair care composition of from about 0.5 microns to about 20 microns.

2. The hair care composition of claim 1 wherein the hair care composition further comprises:
from about 0.1% to about 20% of one or more high melting point fatty compounds, by weight of said hair care composition;
from about 0.1% to about 10% a cationic surfactant system of, by weight of said hair care composition; and
at least about 20% of an aqueous carrier, by weight of said hair care composition; and
wherein the hair care composition comprises from about 0.005% to about 5% of said one or more material, by weight of said hair care composition.

3. The hair care composition of claim 1, wherein the discrete particle has an average particle size in the hair care composition of from about 0.8 microns to about 15 microns.

4. The hair care composition of claim 3, wherein the discrete particle has an average particle size in the hair care composition of from about 1.0 microns to about 10 microns.

5. The hair care composition of claim 4, wherein the discrete particle has an average particle size in the hair care composition of from about 1.0 microns to about 6.0 microns.

6. The hair care composition of claim 1, wherein the oily component has Zero Shear Viscosity at 25° C. of from about 10³ Pa·s to about 10⁸ Pa·s.

7. The hair care composition of claim 6, wherein the oily component has Zero Shear Viscosity at 25° C. of from about 10⁴ Pa·s to about 10⁷ Pa·s.

8. The hair care composition of claim 1 wherein the oily component has a melting point of from about 40° C. to about 52° C.

9. The hair care composition of claim 8 wherein the oily component has a melting point of from about 40° C. to about 50° C.

10. The hair care composition of claim 2, wherein the hair care composition comprises from about 0.01% to about 3% of said one or more materials, by weight of said hair care composition.

11. The hair care composition of claim 10, wherein the hair care composition comprises from about 0.01% to about 2% of said one or more materials, by weight of said hair care composition.

12. The hair care composition of claim 11, wherein the hair care composition comprises from about 0.01% to about 1% of said one or more materials, by weight of said hair care composition.

13. The hair care composition of claim 1, wherein said one or more material is (A) metathesized unsaturated polyol esters.

14. The hair care composition of claim 13, wherein said one or more material is metathesized unsaturated polyol esters selected from oligomers.

15. The hair care composition of claim 14, wherein said oligomer is a triglyceride oligomer.

16. The hair care composition of claim 15, wherein said triglyceride oligomer is a soy oligomer.

17. The hair care composition of claim 1, wherein said oily component further comprises an oily material which is liquid at 25° C.

18. The hair care composition of claim 17, wherein said oily component comprises said one or more material being a metathesized unsaturated polyol ester, and the oily component further comprises oily material which is liquid at 25° C. and which is a non-metathesized unsaturated polyol ester.

19. The hair care composition of claim 18, wherein said oily component comprises said one or more material being a metathesized unsaturated polyol ester being a soy oligomer, and the oily component further comprises oily material which is liquid at 25° C. and which is non-metathesized unsaturated polyol ester being a soybean oil.

20. The hair care composition of claim 1, wherein the discrete particle is pre-emulsified by an emulsifier selected from the group consisting of anionic, non-ionic, cationic, amphoteric emulsifiers and mixtures thereof.

21. The hair care composition of claim 20, wherein the discrete particle is pre-emulsified by a nonionic emulsifier.

22. The hair care composition of claim 21, wherein the nonionic emulsifier is selected from the group consisting of polysorbates.

23. A method of providing hair styling benefit to hair, comprising a step of applying to the hair an effective amount of the hair care composition of claim 1.
